(19) United States
(12) Patent Application Publication
(10) Pub. No.: US 2024/0082479 A1
(43) Pub. Date: Mar. 14, 2024

(54) FLUID DELIVERY SYSTEM CONTAINER AND METHOD OF USE

(71) Applicant: TAKEDA PHARMACEUTICAL COMPANY LIMITED, (US)

(72) Inventors: Jeffrey PELTIER, Richmond, RI (US); Peter MURPHY, Bray, Co. Wicklow (IE); Eugene CANAVAN, Bray, Co. Wicklow (IE)

(73) Assignee: TAKEDA PHARMACEUTICAL COMPANY LIMITED, Osaka (JP)

(21) Appl. No.: 18/039,137

(22) PCT Filed: Nov. 22, 2021
(86) PCT No.: PCT/JP2021/043518

§ 371 Date: May 26, 2023

Related U.S. Application Data
(60) Provisional application No. 63/119,797, filed on Dec. 1, 2020.

(51) Int. Cl. A61M 5/00; A61J 1/20; A61M 5/24

(52) U.S. Cl. CPC A61M 5/002; A61J 1/2089; A61M 5/2448; A61M 5/2455; A61M 2005/2403

(57) ABSTRACT

Containers for one or more fluid delivery systems are disclosed.

### TECHNICAL FIELD

The present disclosure generally relates to containers for fluid delivery systems.

### BACKGROUND ART

The administration of fluid therapeutics often involves mixing substances to form (e.g., reconstitute) the fluid therapeutic (e.g., drug in liquid form) and the subsequent delivery of the liquid drug to the patient. It is understood that reconstitution, as that term is used throughout this disclosure, is the process of mixing a lyophilized active agent with a solvent so the active agent can be re-liquefied, or reconstituted, before injection. The process is common, since lyophilizing (or freeze-drying) an active agent, such as a drug, biologic, or other therapeutic, can help to preserve the shelf life of the agent and enable the agent to be delivered to the end user for future mixing. That said, many times the mixing of the agent is expected to be completed by someone other than the manufacturer or a pharmacist, so care must be taken to ensure that the mixing of the solvent and the active agent is accurate and error-proof.

Injectors that include these fluid therapeutics can come preloaded from the ready to reconstitute to then administer. In such instances, these injectors are sold to patients and caregivers as part of therapeutic “containers.” These containers may include, for example, one or more injectors, disposable needles, swabs, and other the substances that will form the therapeutic liquid, as well as a container for containing these components.

### SUMMARY OF THE INVENTION

The solution of this disclosure resolves these and other issues of the art.

In some examples of the present disclosure, a container is disclosed that includes a closed configuration and an open configuration.

In some examples, a container is disclosed that includes a first member including an outer surface. A second member is included with a substantially planar surface and hingedly connected to the first member. A third member can be included with an outer surface and hingedly connected to the second member, the third member including a substantially cylindrical recess configured to releasably hold a proximal end of a fluid delivery system so that a distal end of the fluid delivery system is oriented in an upright position during and/or after reconstitution, wherein the fluid delivery system in the upright position is orthogonal to one or more of the fluid delivery systems releasably held in retaining grooves of the first and/or third member. The container can be configured such that in a closed configuration, the outer surfaces of the first and third members are substantially parallel with each other with outer edges of the first and third members contacting or immediately adjacent thereto to form an enclosed housing. The container can be configured such that in an open configuration, the outer surfaces of the first, second, and third members are substantially planar to one another and facing the same direction.

In some examples, the second member is positioned between the first and third members.

In some examples, the second member is orthogonal to the first and third members in the closed configuration.

In some examples, the first member is opposite the third member.

In some examples, the first, second, and third members are integrally formed with each other.

In some examples, the substantially planar surface of the second member lacks any structural members to releasably hold anything.

In some examples, the first member includes a plurality of grooves configured to frictionally engage and releasably secure portions of any one of a plurality of fluid delivery systems, each of the plurality of fluid delivery systems being a pen injector.

In some examples, the grooves are configured to releasably hold at least six (6) separate fluid delivery systems.

In some examples, the grooves are configured to releasably hold at least four (4) separate fluid delivery systems.

In some examples, the grooves releasably hold the plurality of fluid delivery systems so that each is aligned with the other and oriented parallel with the outer surface of the second member.

In some examples, the grooves are formed (e.g., injection molded) in a tray coextensive with the first member, the grooves being configured to cradle at least one of the plurality of fluid delivery systems securely in a predetermined position during transport of the case. The tray can be detachable from the first member.

In some examples, the third member can include a plurality of needle recesses configured to releasably hold sealed disposable needles for use with a distal end of the fluid delivery system.

In some examples, during and/or after reconstitution, an active agent and a solvent included in a distal ampoule portion of the fluid delivery system in the upright position mixes for a predetermined period of time.

In some examples, the substantially cylindrical recess is located adjacent a corner of the third member opposite the second member.

In some examples, each fluid delivery system of the container includes an ampoule prepackaged with an active agent and a solvent whereby the ampoule is incapable of being removed from the fluid delivery system by an end user.

In some examples, a container is disclosed that includes a first member including a substantially planar surface. A second member is also included with a substantially planar surface hingedly connected to the first member, the first member including a greater width than the first member. A third member is included with an outer surface and hingedly connected to the second member. The third member can include a plurality of grooves configured to frictionally engage and releasably secure portions of any one of a plurality of fluid delivery systems, each of the plurality of fluid delivery systems being a pen injector. The third member can also include a substantially cylindrical recess configured to releasably hold a proximal end of one of the fluid delivery systems so that a distal end of the fluid delivery system is oriented in an upright position during and/or after reconstitution, and the fluid delivery system in the upright position is orthogonal to one or more of the fluid delivery systems releasably held in the grooves of the third member. The container is configured such that in a closed configuration, the first and third members are substantially parallel with each other with edges of the first and third members contacting or immediately adjacent thereto to form an enclosed housing. The container is configured such that in an open configuration, the outer surfaces of the first, second, and third members are substantially planar to one another and facing the same direction.

In some examples, the second member are positioned between the first and third members.

In some examples, the substantially planar surfaces of the first and second members are continuous with each other.

In some examples, the second member are orthogonal to the first and third members in the closed configuration.

In some examples, the first, second, and third members are integrally formed with each other.

In some examples, the substantially planar surfaces of the first and second members are not configured with any structural members to releasably hold anything (e.g., a flush flat surface with no structural features).

In some examples, the third member includes a plurality of grooves configured to frictionally engage and releasably secure portions of any one of a plurality of fluid delivery systems, each of the plurality of fluid delivery systems being a pen injector.

In some examples, the grooves are configured to releasably hold at least six (6) separate fluid delivery systems.

In some examples, the grooves are configured to releasably hold at least four (4) separate fluid delivery systems.

In some examples, the grooves releasably hold the plurality of fluid delivery systems so that each is aligned with the other and oriented parallel with the outer surface of the third member.

In some examples, the grooves are formed (e.g., injection molded) in a tray coextensive with the third member, the grooves being configured to cradle at least one of the plurality of fluid delivery systems securely in a predetermined position during transport of the case. The tray can be detachable from the third member.

In some examples, during and/or after reconstitution, an active agent and a solvent included in a distal ampoule portion of the fluid delivery system in the upright position mixes for a predetermined period of time.

In some examples, the substantially cylindrical recess is located adjacent a corner of the third member opposite the second member.

In some examples, each fluid delivery system includes an ampoule prepackaged with an active agent and a solvent whereby the ampoule is incapable of being removed from the fluid delivery system by an end user.

In some examples, the third member includes one or more grooves configured to frictionally engage and releasably secure portions of a fluid delivery system, the fluid delivery system being a pen injector.

In some examples, the grooves are formed (e.g., injection molded) in a tray coextensive with the third member, the grooves being configured to cradle the fluid delivery system securely in a predetermined position during transport of the case. The tray can be detachable from the third member.

In some examples, the third member includes a substantially cylindrical recess configured to releasably hold a proximal end of the fluid delivery system so that a distal end of the fluid delivery system is oriented in an upright position during and/or after reconstitution, wherein the fluid delivery system in the upright position is orthogonal to its position in the closed configuration when releasably held in the grooves of the third member.

In some examples, during and/or after reconstitution, an active agent and a solvent included in a distal ampoule portion of the fluid delivery system in the upright position mixes for a predetermined period of time.

In some examples, the substantially cylindrical recess is located adjacent a corner of the third member opposite the second member.

In some examples, the fluid delivery system includes an ampoule prepackaged with an active agent and a solvent whereby the ampoule is incapable of being removed from the fluid delivery system by an end user.

In some examples, the fluid delivery system of this disclosure includes a housing, a cartridge holder fixedly mechanically coupled to the housing, a cartridge disposed within a cartridge holder internal cavity. The cartridge can include a distal tip; a first stopper; a second stopper disposed between the distal tip and the first stopper; an active agent disposed between the distal tip and the second stopper; and a solvent disposed between the second stopper and the first stopper.

In some examples, the active agent includes lyophilized teduglutide.

In some examples, a method is disclosed that can include causing an active agent of a fluid delivery system in any container of this disclosure to mix with a solvent disposed in the fluid delivery system thereby creating a reconstituted liquid. The method can include positioning a proximal end of the fluid delivery system in a substantially cylindrical recess of the container for a predetermined period of time during and/or after reconstitution.

In some examples, the method can include positioning one or a plurality of fluid delivery systems within a recess or one or more grooves of the first and/or third member of the container.

In some examples, the method can include a step of causing, by a user, the container to move from a closed configuration to the opened configuration.

In some examples, the method can include a step of causing, by a user, the container to move from an open configuration to a closed configuration.

In some examples, the method can include a step of selecting, by a user, one of a plurality of fluid delivery systems contained in the container, where each of the plurality of fluid delivery systems comprises medication corresponding to one or more days of treatment.

In some examples, the method can include a step of positioning a predetermined number of the plurality of fluid delivery systems in the container for a predetermined duration of treatment. The predetermined duration of treatment can be approximately a week and the predetermined number being at least four (4), six (6), or any greater or lower number of fluid delivery systems.

In some examples, the method can, include a step of positioning a plurality of containers within a housing of a kit, the plurality of containers collectively comprising approximately one (1) month of medication.

In some examples, the method can include selecting one of the plurality of containers; delivering medication, by the fluid delivery systems of the selected container to the patient; upon use of each of the plurality of fluid delivery systems of the selected container, detaching the first or third member previously comprising the plurality of fluid delivery systems; and then attaching a first or third member with another of the plurality of containers to the selected container.

In some examples, the method can include selecting one of the plurality of containers; delivering medication, by the fluid delivery systems of the selected container to the patient; upon use of each of the plurality of fluid delivery systems of the selected container, detaching the first or third member previously comprising the plurality of fluid delivery systems; and then attaching a first or third member with another of the plurality of containers to the selected container.

In some examples, the step of causing the active agent to mix with the solvent can include rotating the cartridge holder with respect to the housing causing the cartridge holder to move axially proximally through the housing from a first position to a second position; and rotating the cartridge holder with respect to the housing causing a button rod to move axially distally through the cartridge.

In some examples, the active agent can be lyophilized teduglutide. However, other active agents are contemplated as needed or required.

In some examples, a kit is disclosed that includes one or more containers according to this disclosure.

In some examples, each container in the kit includes a predetermined supply of fluid delivery systems for a predetermined duration of treatment.

In some examples, each fluid delivery system of the kit includes approximately 3 ml of medication comprised by the active agent and the solvent.

In some examples, each fluid delivery system of the kit includes approximately 4 ml of medication comprised by the active agent and the solvent.

In some examples, the kit is configured to provide a predetermined medication supply for a predetermined duration of treatment (e.g., approximately a month of medication). For example, the kit can include at least four containers and each container of the kit approximately a medication supply of approximately a week.

To the accomplishment of the foregoing and related ends, certain illustrative aspects are described herein in connection with the following description and the appended drawings. These aspects are indicative, however, of but a few of the various ways in which the principles of the claimed subject matter can be employed and the claimed subject matter is intended to include all such aspects and their equivalents. Other advantages and novel features can become apparent from the following detailed description when considered in conjunction with the drawings.

### BRIEF DESCRIPTION OF THE DRAWINGS

The above and further aspects of this disclosure are further discussed with reference to the following description in conjunction with the accompanying drawings, in which like numerals indicate like structural elements and features in various figures. The drawings are not necessarily to scale, emphasis instead being placed upon illustrating principles of the disclosure. The figures depict one or more implementations of the inventive devices, by way of example only, not by way of limitation.

FIG. 1 is a view of a container according to an example of the present disclosure.

FIG. 2A is an upper perspective view of a container in an open configuration according to an example of the present disclosure.

FIG. 2B is an upper perspective view of the container of FIG. 2A in an open configuration including various components according to an example of the present disclosure.

FIG. 2C is an upper perspective view of the container of FIG. 2A in a closed configuration according to an example of the present disclosure.

FIG. 3A is an upper perspective view of a container in an open configuration according to an example of the present disclosure.

FIG. 3B is an upper perspective view of the container of FIG. 3A in an open configuration including various components according to an example of the present disclosure.

FIG. 3C is an upper perspective view of the container of FIG. 3A in a closed configuration according to an example of the present disclosure.

FIG. 4A is an upper perspective view of a container in an open configuration according to an example of the present disclosure.

FIG. 4B is an upper perspective view of the container of FIG. 4A in an open configuration including various components according to an example of the present disclosure.

FIG. 4C is an upper perspective view of the container of FIG. 4A in a closed configuration according to an example of the present disclosure.

FIG. 5A is an upper perspective view of a container in an open configuration according to an example of the present disclosure.

FIG. 5B is an upper perspective view of a container in a partially open configuration according to an example of the present disclosure.

FIG. 5C is an upper perspective view of a container in a partially open configuration according to an example of the present disclosure.

FIG. 5D is an upper perspective view of a container in a partially open configuration according to an example of the present disclosure.

FIG. 6 depict a method of using any container of this disclosure (e.g., a pen injector prepackaged with an active agent mixable with a solvent for delivery to a patient).

### DETAILED DESCRIPTION

Although examples of the disclosed technology are explained in detail herein, it is to be understood that other examples are intended to be within the scope of the claimed invention. Accordingly, it is not intended that the disclosed technology be limited in its scope to the details of construction and arrangement of components set forth in the following description or illustrated in the drawings. The disclosed technology is capable of other examples and of being practiced or carried out in various ways.

It must also be noted that, as used in the specification and the appended claims, the singular forms “a,” “an” and “the” include plural referents unless the context clearly dictates otherwise. By “comprising” or “containing” or “including” it is meant that at least the named compound, element, particle, or method or use step is present in the composition or article or method or use, but does not exclude the presence of other compounds, materials, particles, method or use steps, even if the other such compounds, material, particles, method or use steps have the same function as what is named.

As used herein, the terms “about” or “approximately” for any numerical values or ranges indicate a suitable dimensional tolerance that allows the part or collection of components to function for its intended purpose as described herein. More specifically, “about” or “approximately” can refer to the range of values ±20% of the recited value, e.g. “about 90%” can refer to the range of values from 71% to 99%.

In addition, as used herein, the terms “patient,” “host,” “user,” and “subject” refer to any human or animal subject and are not intended to limit the systems or method or uses to human use, although use of the subject invention in a human patient represents a preferred example.

As used herein, the term “drug” or “medication” refers to a liquid therapeutic used in a fluid delivery system, such as a pen injector (e.g., an autoinjector), pertaining to the treatment or curing of a disease or medical condition. Such medications may involve, for example, a mix of one part solvent with one part active ingredient that requires reconstitution prior to delivery to a patient, whereby the medication is intended to treat a disease or medical condition, including but not limited to irritable bowel syndrome, though other conditions (e.g., diabetes) treatable with a fluid delivery system of this disclosure is contemplated.

In describing examples, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method or use does not preclude the presence of additional method or use steps or intervening method or use steps between those steps expressly identified. Steps of a method or use can be performed in a different order than those described herein without departing from the scope of the disclosed technology. Similarly, it is also to be understood that the mention of one or more components in a device or system does not preclude the presence of additional components or intervening components between those components expressly identified.

The present disclosure is related to containers for one or more fluid delivery systems (e.g., autoinjectors or pen injectors) and related method or uses. The present disclosure will now be described with reference to examples as illustrated in the accompanying drawings. References to “one example”, “an example”, “an example”, or the like indicate that the example described may include a particular feature, structure, or characteristic, but every example may not necessarily include the particular feature, structure, or characteristic. Moreover, such phrases are not necessarily referring to the same example.

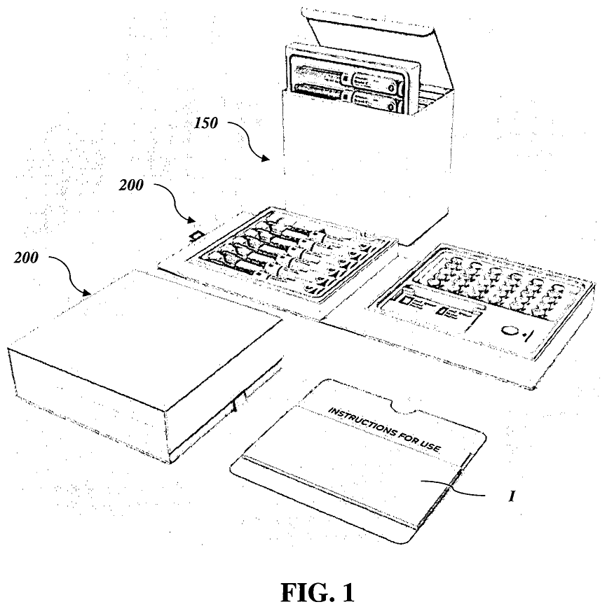

FIG. 1 is a front perspective view from above of a kit 150 that includes an example container 200 slidably stored upright in an open kit. Another example container 200 storable in kit 150 is shown adjacent kit 150 having been withdrawn therefrom and placed on a treatment surface in an open configuration. In the open configuration, example components stored therein are shown (e.g., injectors, disposable needle caps N, and instructions I of use, etc.). Kit 150 is particularly useful to patients and the like because they can include all necessary components for a patient to self-administer a subset of their treatment and contained in a single container, such as container 200. Container 200 can broadly be understood as anything configured to contain one or more autoinjectors with drug pre-loaded therein, such as a carton or box. Containers 200 as shown more particularly below can come in a variety of shapes and sizes, be made from a variety of materials, and have a variety of open and closed configurations.

While container 200 may be generally rectangular, the container shape is not so limited can it is contemplated that containers of this disclosure can include numerous shapes, such as a cube, being triangular, spherical, cylindrical, conical, or any other shape as needed or required. Kit 150 and/or containers 200 may be made from a wide variety of materials. For example, kit 150 and/or containers 200 can be constructed as a composite or monolithic structure with plastics (e.g., injection molded polypropylene, polyvinylchloride, polytetrafluoroethylene, polyether sulfone, polyethylene, polyurethane, polyetherimide, polycarbonate, polyetheretherketone, polysulfone, cyclic olefin polymer, or cyclic olefin copolymer) as well as other materials such as cardboard, paper, metal, glass, wood, and/or the like. As shown, kit 150 can be configured to contain multiple containers 200 (e.g., five (5)) that are slidably inserted through the open upper end into drawer-like compartments. However, containers 200 can also include mechanisms for attaching each container 200 in kit 150 to each other (e.g., snap fit connectors, hook and loop fasteners, magnetic connectors and receivers, etc.).

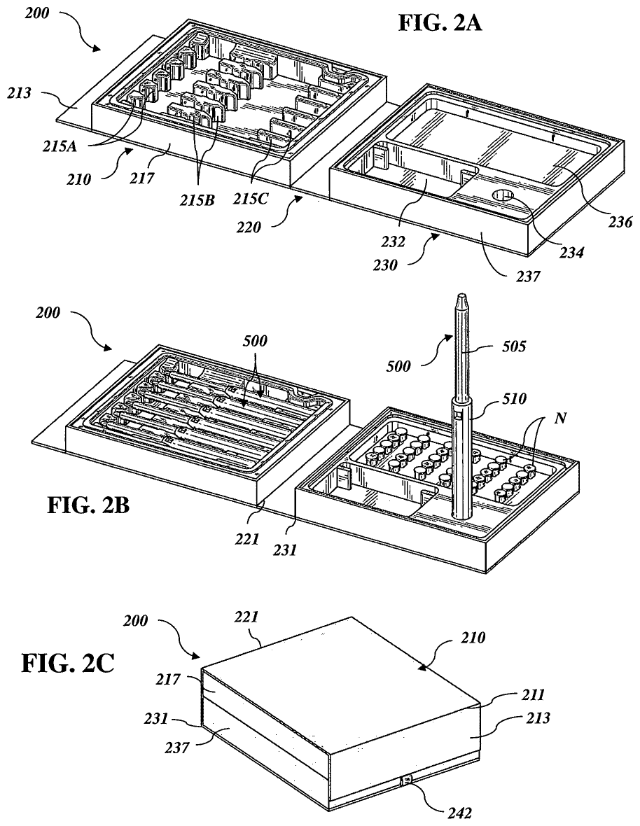

Turning to FIGS. 2A-2C, container 200 is shown in various configurations.

For example, FIG. 2A shows container 200 in an open configuration without any external components, such as fluid delivery system 500 (e.g., a pen injector), needle caps N, alcohol swabs, or any other features necessary for administration of therapeutics from system 500 (including various components). System 500 can be an autoinjector or pen injector prepackaged with medicaments for administration by a care provider or self-administration by a patient, whereby such systems 500 can include one or more features more clearly described in Appendix 1 as incorporated by reference in its entirety from U.S. Provisional application 63/117,538 filed Nov. 24, 2020, which is incorporated by reference in its entirety as if set forth verbatim herein. It is understood that system 500 does need, nor is it capable mechanically, of being separated from its constituent features to introduce or change medication prepackaged therein. For example, no separate vial of some separate or additional medication (e.g., a solvent or active ingredient such as lyophilized teduglutide) is needed or usable with system 500, since system 500 of this disclosure and used with the various containers and systems herein already includes all necessary medication. The system 500 of this disclosure can include approximately 3 ml, approximately 4 ml, and/or the like as defined by the active agent and the solvent.

Container 200 can include a plurality of members 210, 220, 230. For example, container 200 can include first member 210 hingedly connected to second member 220, and third member 230 hingedly connected to second member 220. However, container 200 is not so limited and greater or fewer number of members can be included in container 200 as needed or required. Members 210, 230 can include substantially planar lower surfaces configured to rest on treatment surfaces with one or more sidewalls opposite thereto and other structural members.

Member 210 can include one or more sidewalls 217 extended upward, away from the outer surface. The one or more sidewalls 217 can form a cavity within which one or more grooves 215A, 215B, 215C can be positioned to releasably hold one or more systems 500. Each of grooves 215A, 215B, 215C can be configured to releasably hold portions of system (e.g., groove 215A can be positioned to releasably hold by frictional engagement distal portion 505 of system 500). Greater or fewer grooves 215A, 215B, 215C can be included as needed. Grooves 215A, 215B, 215C can also be sequentially spaced so that corresponding systems are likewise spaced apart and arranged in sequence. In some examples, grooves 215A, 215B, 215C can be molded into an integrally formed tray detachable from the one or more sidewalls 217.

Member 230 can include one or more sidewalls 237 extended upward, away from the outer surface. The one or more sidewalls 237 can form one or more cavities 236, 232 within which one or more components for use with systems 500 can be temporarily stored (e.g., disposable needle caps N, alcohol swabs, etc.). A substantially cylindrical recess 234 can be positioned between one or more sidewalls 237 on a planar surface. Recess 234 can be configured to releasably hold one of systems 500 upright by the proximal portion 510 so that distal portion 505 is oriented in an upright position during and/or after reconstitution. In the upright position shown in FIG. 2B, system 500 is oriented orthogonal to one or more of the systems 500 releasably held in grooves 215A, 215B, 215C. System 500 in the upright position is also oriented orthogonal to the outer surface of container 200 when positioned in the open configuration on a flat preparation surface.

In contrast to members 210, 230, member 220 is substantially planar lacking any outwardly extending structural members. Rather, member 220 is hingedly connected to member 210 by hinged mechanism 221 and hingedly connected to member 230 by hinged mechanism 231. As shown, each of mechanisms 221, 231 are formed by folds. However, other pivotable mechanisms or hinges are contemplated as needed or required.

With reference to FIG. 2B, container 200 is shown with a plurality of systems 500 arranged in grooves 215A, 215B, 215C of member 210. Each of systems 500 is prepackaged with medication and awaiting reconstitution for delivery to the patient. A user may select any one of systems 500 from member 210 and then position the selected system 500 so that a proximal portion 510 of the system 500 is positioned within recess 234 of third member 230 during and/or after reconstitution.

In some examples, prior to positioning portion 510 within recess 234, the user can prime system 500 by rotating a cartridge holder with respect to the housing of system 500 causing the cartridge holder to move axially proximally through the housing from a first position to a second position. The user can continue rotating the cartridge holder with respect to the housing causing a button rod to move axially distally through the cartridge thereby causing an active agent disposed in distal portion 505 of system 500 between the distal tip of the cartridge and the second stopper to mix with the solvent disposed between the second stopper and the first stopper. In turn, a reconstituted liquid is created in system 500. At this point, the proximal portion 510 (e.g., a button end of system 500) can be positioned within recess 234 for a predetermined period of time until ready for injecting in the patient. It is understood that portions 505, 510 in some examples are permanently attached insofar as being undetachable from each other once in container 200 prior to receipt by the end user.

Container 200 can include six (6) injector systems 500 in member 210, whereby each system 500 collectively including medication for approximately a week of treatment (e.g., each system 500 can include sufficient medication for a day). In turn, since container 200 can include sufficiently daily medication for approximately a week, the kit 150 shown in FIG. 1 can be deliverable to an end user to easily manage a month's supply of medication for a given health condition. It is understood that member 210 can also be detached from container 200 after a user has exhausted the supply of systems 500 (e.g., at the close of a week) and may attach a member 210 from another of containers 200 stored temporarily in kit 150. Of course, kit 150 can include greater or fewer amounts of containers 200 to accommodate greater or fewer durations of treatment time (e.g., if more than a month's supply of medication is desired).

FIG. 2C shows container 200 in a closed configuration where only the outer surfaces are shown. A planar surface 213 can extend from member 210 previously shown in FIG. 2A, opposite from member 230. A hinge or fold 211 can exist where surface 213 extends from member 210 so that after or as, member 210 pivots about hinges 221, 231 to close around the opposite sidewalls 237 of member 230, as shown in FIG. 2C. Optionally, a tab 242 can extend from surface 213, as shown clearly in FIG. 2C, to facilitate a user moving container 200 between opened and closed configurations. Tab 242 can couple to a receiver (not shown) on container 200, including a snap receiver or magnetic receiver or any other detachable fastener to facilitate quick release yet secure fastening. As shown, member 210 can be the upper half of container 200 in the closed configuration, while member 230 can be the lower half. The upper surfaces formed by walls 217, 237 can contact each other to form an internal cavity in the closed configuration. Member 220 in FIG. 2C is not shown but forms the aft book hinge wall bridging together members 210, 230.

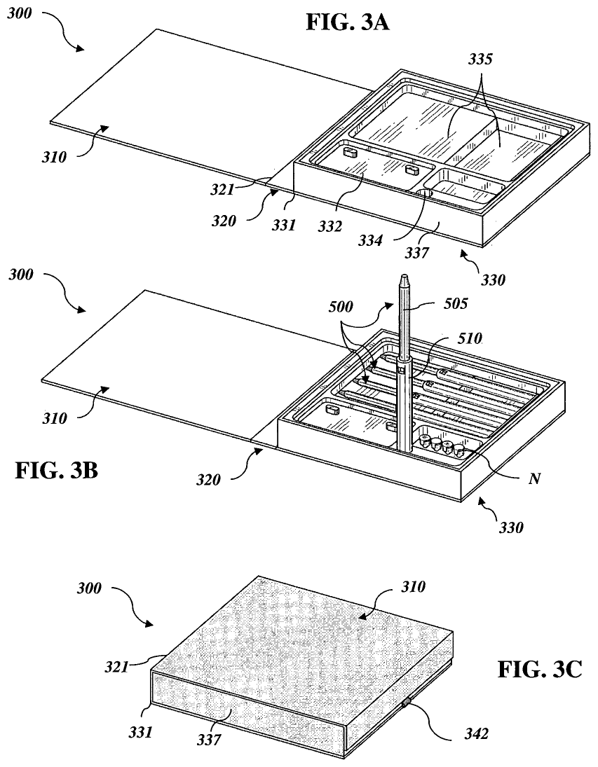

Turning to FIGS. 3A-3C, container 300 is shown in various configurations. For example, FIG. 3A shows container 300 in an open configuration without any external components, such as system 500, needle caps N, alcohol swabs, or any other features necessary for administration of therapeutics from system 500 (including various components). Container 300 can include a plurality of members 310, 320, 330. For example, container 300 can include first member 310 hingedly connected to second member 320, and third member 330 hingedly connected to second member 320. However, container 300 is not so limited and greater or fewer number of members can be included in container 300 as needed or required. Members 310, 330 can include substantially planar lower surfaces configured to rest on treatment surfaces.

Member 330 can include one or more sidewalls 337 extended upward, away from the outer surface. The one or more sidewalls 337 can form one or more cavities 335, 332, 333 within which one or more of systems 500 or components for use with systems 500 can be temporarily stored (e.g., disposable needle caps N, alcohol swabs, etc.). A substantially cylindrical recess 334 can be positioned between one or more sidewalls 337 on a planar surface (e.g., adjacent one of cavities 335, 332, 333). Recess 334 can be configured to releasably hold one of systems 500 upright by the proximal portion 510 so that distal portion 505 is oriented in an upright position during and/or after reconstitution. In the upright position shown in FIG. 3B, system 500 is oriented orthogonal to one or more of the systems 500 previously had while releasably held in cavities 335, 332, 333. System 500 in the upright position is also oriented orthogonal to the outer surface of container 300 when positioned in the open configuration on a flat preparation surface.

In contrast to member 330, members 310, 320 are both substantially planar, lacking any outwardly extending structural members. Rather, member 320 is hingedly connected to member 310 by hinged mechanism 321 and hingedly connected to member 330 by hinged mechanism 331. As shown, each of mechanisms 321, 331 are formed by folds. However, other pivotable mechanisms or hinges are contemplated as needed or required. With reference to FIG. 3B, container 300 is shown with a plurality of systems 500 arranged in cavities 335 of member 330. Each of systems 500 is prepackaged with medication, including necessary solvent and active ingredient in each system 500, and awaiting reconstitution for delivery to the patient. A user may select any one of systems 500 from member 330 and then position the selected system 500 so that a proximal portion 510 of the system 500 is positioned within recess 334 of third member 330 during and/or after reconstitution, as previously discussed.

FIG. 3C shows container 300 in a closed configuration where only the outer surfaces are shown. In the closed configuration, tab 342 can extend from surface 313, as shown clearly in FIG. 3C, to facilitate a user moving container 300 between opened and closed configurations. As shown, the upper surfaces formed by walls 337 can contact the inner surface of 310 to form an internal cavity in the closed configuration. Member 320 in FIG. 3C is not shown but forms the aft book hinge wall bridging together members 310, 330.

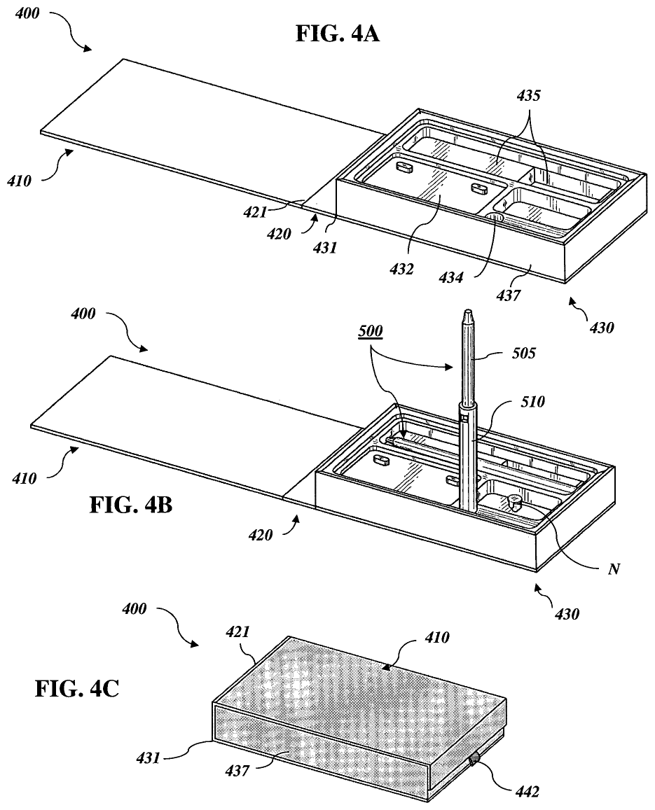

Turning to FIGS. 4A-4C, container 400 is shown in various configurations. For example, FIG. 4A shows container 400 in an open configuration without any external components, such as system 500, needle caps N, alcohol swabs, or any other features necessary for administration of therapeutics from system 500 (including various components). Container 400 can include a plurality of members 410, 420, 430. For example, container 400 can include first member 410 hingedly connected to second member 420, and third member 430 hingedly connected to second member 420. However, container 400 is not so limited and greater or fewer number of members can be included in container 400 as needed or required. Members 410, 430 can include substantially planar lower surfaces configured to rest on treatment surfaces.

Member 430 can include one or more sidewalls 437 extended upward, away from the outer surface. The one or more sidewalls 437 can form one or more cavities 435, 432, 433. Container 400 is configured to house only one system 500 in cavity container 435, as shown in FIG. 4B. However, ancillary components that support use with system 500 can be temporarily stored in cavities 433, 432 (e.g., disposable needle caps N, alcohol swabs, etc.). A substantially cylindrical recess 434 can be positioned between one or more sidewalls 437 on a planar surface (e.g., adjacent one of cavities 435, 432, 433). Recess 434 can be configured to releasably hold system 500 upright by the proximal portion 510 so that distal portion 505 is oriented in an upright position during and/or after reconstitution. In the upright position shown in FIG. 4B, system 500 is oriented orthogonal to the orientation system 500 previously had while releasably held in cavity 435. System 500 in the upright position is also oriented orthogonal to the outer surface of container 400 when positioned in the open configuration on a flat preparation surface.

In contrast to member 430, members 410, 420 are both substantially planar lacking any outwardly extending structural members. Rather, member 420 is hingedly connected to member 410 by hinged mechanism 421 and hingedly connected to member 430 by hinged mechanism 431. As shown, each of mechanisms 421, 431 are formed by folds. However, other pivotable mechanisms or hinges are contemplated as needed or required.

With reference to FIG. 4B, container 400 is shown with only one system 500 arranged in cavity 435 of member 430. FIG. 4C shows container 400 in a closed configuration where only the outer surfaces are shown. In the closed configuration, tab 442 can extend from surface 413, as shown clearly in FIG. 4C, to facilitate a user moving container 400 between opened and closed configurations. As shown, the upper surfaces formed by walls 437 can contact the inner surface of 410 to form an internal cavity in the closed configuration. Member 420 in FIG. 4C is not shown but forms the aft book hinge wall bridging together members 410, 430.

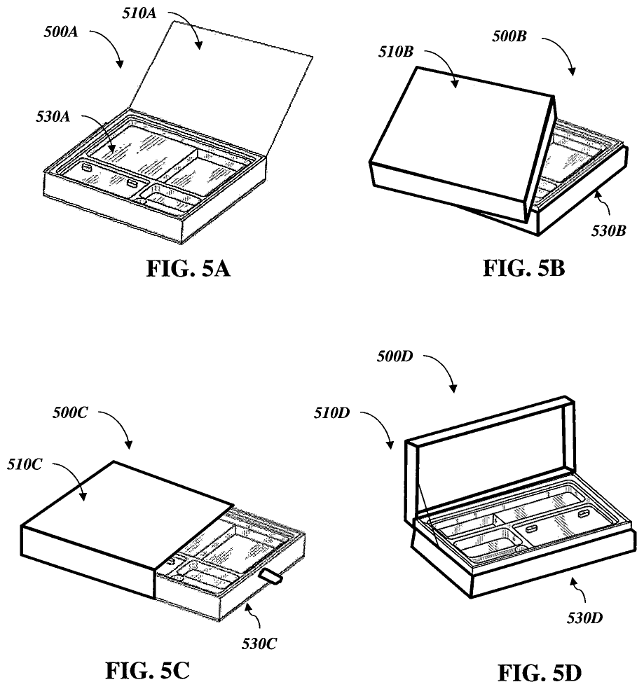

Turning to FIGS. 5A-5D, additional embodiments of containers shown in various configurations. For example, FIG. 5A shows container 500A in an open configuration without any external components, such as system 500, needle caps N, alcohol swabs, or any other features necessary for administration of therapeutics from system 500 (including various components). It can be seen that container 500A includes a substantially planar member 510A hingedly connected to member 530A, which includes features similar to previously described member 330. In the closed configuration, member 510A can pivot about a shared axis to close over member 530A.

FIG. 5B shows container 500B in a partially open configuration without any external components, such as system 500, needle caps N, alcohol swabs, or any other features necessary for administration of therapeutics from system 500 (including various components). It can be seen that container 500B includes a detachable member 510B that can fit over member 530B, which can include features similar to previously described member 330. Member 530B can include a step surface extended outward from its sidewalls on which member 510B can snugly rest in the closed configuration, similar to a shoe box.

FIG. 5C shows container 500C in a partially open configuration without any external components, such as system 500, needle caps N, alcohol swabs, or any other features necessary for administration of therapeutics from system 500 (including various components). It can be seen that container 500C includes member 510C which can include a single open end through which member 530C can be slid into and out in a drawer-like manner. Member 530C can include features similar to previously described member 330.

FIG. 5D shows container 500D in an open configuration without any external components, such as system 500, needle caps N, alcohol swabs, or any other features necessary for administration of therapeutics from system 500 (including various components). It can be seen that container 500D includes a lid member 510D hingedly connected to member 530D, which includes features similar to previously described member 430. In the closed configuration, member 510D can pivot to close over member 530D.

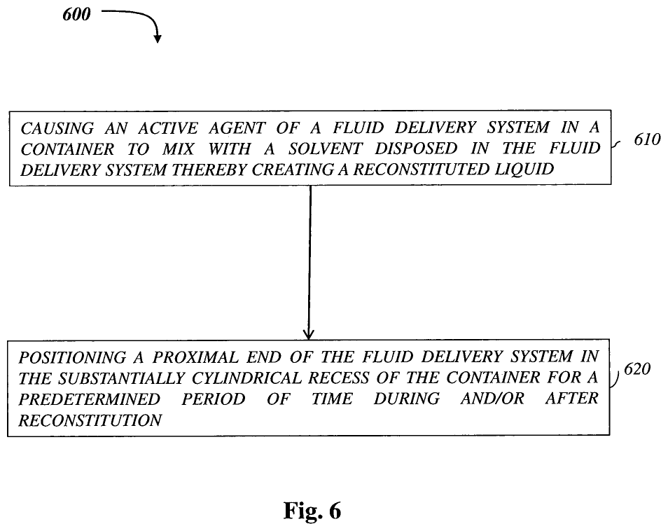

FIG. 6 depicts a method 600 is disclosed. The method 600 can include 610 causing an active agent of a fluid delivery system in any container of this disclosure to mix with a solvent disposed in the fluid delivery system thereby creating a reconstituted liquid. The method 600 can include 620 positioning a proximal end of the fluid delivery system in a substantially cylindrical recess of the container for a predetermined period of time during and/or after reconstitution.

It is to be understood that the mention of one or more steps of a method, including method 600, does not preclude the presence of additional method steps or intervening method steps between those steps expressly identified. Steps of a method can be performed in a different order than those described herein without departing from the scope of the disclosed technology.

In this respect, method 600 can include positioning one or a plurality of fluid delivery systems within a recess or one or more grooves of the first and/or third member of the container.

The method 600 can also include a step of causing, by a user, the container to move from a closed configuration to the opened configuration.

The method 600 can also include a step of causing, by a user, the container to move from an open configuration to a closed configuration.

The method 600 can also include a step of selecting, by a user, one of a plurality of fluid delivery systems contained in the container, where each of the plurality of fluid delivery systems comprises medication corresponding to one or more days of treatment.

The method 600 can also include a step of positioning a predetermined number of the plurality of fluid delivery systems in the container for a predetermined duration of treatment. The predetermined duration of treatment can be approximately a week and the predetermined number being at least four (4), six (6), or any greater or lower number of fluid delivery systems.

The method 600 can also include a step of positioning a plurality of containers within a housing of a kit, the plurality of containers collectively comprising approximately one (1) month of medication.

The method 600 can also include selecting one of the plurality of containers; delivering medication, by the fluid delivery systems of the selected container to the patient; upon use of each of the plurality of fluid delivery systems of the selected container, detaching the first or third member previously comprising the plurality of fluid delivery systems; and then attaching a first or third member with another of the plurality of containers to the selected container.

The method 600 can include selecting one of the plurality of containers; delivering medication, by the fluid delivery systems of the selected container to the patient; upon use of each of the plurality of fluid delivery systems of the selected container, detaching the first or third member previously comprising the plurality of fluid delivery systems; and then attaching a first or third member with another of the plurality of containers to the selected container. In some examples, in method 600 the step of causing the active agent to mix with the solvent can include rotating the cartridge holder with respect to the housing causing the cartridge holder to move axially proximally through the housing from a first position to a second position; and rotating the cartridge holder with respect to the housing causing a button rod to move axially distally through the cartridge.

In describing example examples, terminology is resorted to for the sake of clarity. It is intended that each term contemplates its broadest meaning as understood by those skilled in the art and includes all technical equivalents that operate in a similar manner to accomplish a similar purpose. The descriptions contained herein are examples of examples of the disclosure and are not intended in any way to limit the scope of the disclosure.

While particular examples of the present disclosure are described, various modifications to devices and methods can be made without departing from the scope and spirit of the disclosure. For example, while the examples described herein refer to particular components of a container, the disclosure includes other examples utilizing various combinations of components to achieve a described functionality, utilizing alternative materials to achieve a described functionality, combining components from the various examples, combining components from the various example with known components, etc. The disclosure contemplates substitutions of component parts illustrated herein with other well-known and commercially-available products. To those having ordinary skill in the art to which this disclosure relates, these modifications are often apparent and are intended to be within the scope of the claims which follow.

1. A container, comprising:
a first member comprising an outer surface and a plurality of grooves opposite the outer surface and configured to frictionally engage and releasabaly secure portions of any one of a plurality of fluid delivery systems, each of the plurality of fluid delivery systems being a pen injector;
a second member comprising a substantially planar surface and hingedly connected to the first member; and
a third member comprising an outer surface and hingedly connected to the second member, the third member comprising a substantially cylindrical recess configured to releasably, hold a proximal end of one of the plurality of fluid delivery systems so that a distal end of the fluid delivery system is oriented in an upright position during and/or after reconstitution, wherein the fluid delivery system in the upright position is orthogonal to one or more of the plurality of fluid delivery systems releasably held in the grooves
wherein the container is configured such that in a closed configuration, the outer surfaces of the first and third members are substantially parallel with each other with outer edges of the first and third members contacting or immediately adjacent thereto to form an enclosed housing; and
wherein the container is configured such that in an open configuration, the outer surfaces of the first, second, and third members are substantially planar to one another and facing the same direction.

2. The container of claim 1, the second member being positioned between the first and third members.

3.-8. (canceled)

9. The container of claim 1, wherein the grooves are configured to releasably hold at least four (4) separate fluid delivery systems.

10. The container of claim 1, wherein the grooves releasably hold the plurality of fluid delivery systems so that each is aligned with the other and oriented parallel with the outer surface of the second member.

11. The container of claim 1, wherein the grooves are formed in a tray coextensive with the first member, the grooves being configured to cradle at least one of the plurality of fluid delivery systems securely in a predetermined position during transport of the container.

12. The container of claim 1, wherein the third member comprises:
a plurality of needle recesses configured to releasably hold sealed disposable needles for use with a distal end of the fluid delivery system.

13. The container of claim 12, whereby, during and/or after reconstitution, an active agent and a solvent comprised in a distal ampoule portion of the fluid delivery system in the upright position mixes for a predetermined period of time.

14. The container of claim 12, wherein the substantially cylindrical recess is located adjacent a corner of the third member opposite the second member.

15. The container of claim 1, wherein each fluid delivery system comprises an ampoule prepackaged with an active agent and a solvent whereby the ampoule is incapable of being removed from the fluid delivery system by an end user.

16.-34. (canceled)

35. The container of claim 1, wherein the fluid delivery system comprises:
a housing;
a cartridge holder fixedly coupled to the housing;
a cartridge disposed within a cartridge holder internal cavity, the cartridge comprising:
a distal tip;
a first stopper;
a second stopper disposed between the distal tip and the first stopper;
an active agent disposed between the distal tip and the second stopper; and
a solvent disposed between the second stopper and the first stopper.

36. The container of claim 35, wherein the active agent comprises lyophilized teduglutide.

37.-38. (canceled)

39. A method, comprising:
positioning a predetermined number of the plurality of fluid delivery systems in the container of Claim 35 for a predetermined duration of treatment;
selecting, by a user, one of the plurality of fluid delivery systems, where each of the plurality of fluid delivery systems comprises medication corresponding to one or more days of treatment:
causing an active agent of a fluid delivery system in the container to mix with a solvent disposed in the fluid delivery system thereby creating a reconstituted liquid;
positioning a proximal end of the fluid delivery system in a substantially cylindrical recess of the container for a predetermined period of time during and/or after reconstitution;
delivering medication, by the selected one of the fluid delivery, to the patient;
upon use of each of the plurality of fluid delivery systems of the container, detaching the first or third member previously comprising the plurality of fluid delivery systems; and then
attaching another first or third member to the container.

40. The method of claim 39, wherein the active agent comprises lyophilized teduglutide.

41.-46. (canceled)

47. The method of claim 39, the predetermined duration of treatment being approximately a week and the predetermined number being at least four (4) fluid delivery systems.

48.-52. (canceled)

53. A kit comprising one or more containers according to claim 36.

54. The kit of claim 53, wherein each container comprises a predetermined supply of fluid delivery systems for a predetermined duration of treatment.

55. The kit of claim 54, wherein each fluid delivery system comprises approximately 3 ml of medication comprised by the active agent and the solvent.

56. The kit of claim 54, wherein each fluid delivery system comprises approximately 4 ml of medication comprised by the active agent and the solvent.

57. The kit of claim 54, wherein the predetermined duration of treatment is approximately a month, the kit comprising at least four containers and each container comprised by the kit comprises approximately supply to last a week.

58. The container of claim 11, wherein the tray is detachable from the first member and replaceable with a secondary tray with a second plurality of fluid delivery systems.
